(19) United States
(12) Patent Application Publication
(10) Pub. No.: US 2007/0072193 A1
(43) Pub. Date: Mar. 29, 2007

(54) Ligand arrays having controlled feature size, and methods of making and using the same

(76) Inventor: Manish M. Shah, San Jose, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/237,395

(22) Filed: Sep. 27, 2005

(51) Int. Cl. C12Q 1/68; G01N 33/53; C12M 3/00

(52) U.S. Cl. 435/6; 435/7.1; 435/287.2

(57) ABSTRACT

Methods and compositions for producing a solid support having a ligand immobilized on a surface thereof, e.g. a ligand array, are provided. Aspects of the methods include: providing a solid support having a bounded feature location on a surface thereof, where the bounded feature location includes a region of the surface at least partially bounded by an electromagnetic radiation modified boundary; and producing a ligand in the feature location. Also provided are systems for practicing the subject methods, as well as devices produced by the methods and methods of using such devices.

### BACKGROUND OF THE INVENTION

Arrays of binding agents (ligands), such as nucleic acids and polypeptides, have become an increasingly important tool in the biotechnology industry and related fields. These binding agent or ligand arrays, in which a plurality of binding agents are positioned on a solid support surface in the form of an array or pattern, find use in a variety of applications, including gene expression analysis, drug screening, nucleic acid sequencing, mutation analysis, and the like.

A feature of many arrays that have been developed is that each of the polymeric compounds of the array is stably attached to a discrete location on the array surface (referred to in the art as a feature), such that its position remains constant and known throughout the use of the array. Stable attachment is achieved in a number of different ways, including covalent bonding of the polymer to the support surface and non-covalent interaction of the polymer with the support surface.

Where the ligands of the arrays are polymeric, e.g., as is the case with nucleic acid and polypeptide arrays, there are two main ways of producing such arrays, i.e., via deposition of the full ligand, e.g., a pre-synthesized nucleic acid, polypeptide, cDNA fragment, etc., onto the surface of the array; and via in-situ synthesis in which the polymeric ligand is grown on the surface of the substrate in a step-wise fashion.

Methods of depositing obtained biopolymers include dispensing droplets to a substrate from dispensers, such as pin or capillary dispensers (such as described in U.S. Pat. No. 5,807,522) or pulse jet dispensers (such as a piezoelectric inkjet head, as described in PCT publications WO 95/25116 and WO 98/41531, and elsewhere). For in situ fabrication methods, multiple different reagent droplets are deposited from drop dispensers at a given target location in order to form a final feature, for instance, a probe of the feature that is synthesized on the array substrate. The in situ fabrication methods include those described in U.S. Pat. Nos. 5,449,754 and 6,180,351 as well as published PCT publicaton no. WO 98/41531 and the references cited therein.

In array fabrication, the quantities of the biopolymer available, whether by deposition of previously obtained biopolymers or by in situ synthesis, are usually very small and expensive. These conditions require use of arrays with large numbers of very small, closely spaced features. It is important in such arrays that features be deposited accurately in the desired target pattern, and are of the correct size. Failure to meet such quality requirements can have serious consequences to diagnostic, screening, gene expression analysis or other purposes for which the array is being used. However, for economical mass production of arrays with many features it is desirable that they can be fabricated in a short time while maintaining quality. Hence, there is continued interest in the development of new methods for producing ligand arrays.

### SUMMARY OF THE INVENTION

Methods and compositions for producing a solid support having a ligand immobilized on a surface thereof, e.g. a ligand array, are provided. Aspects of the methods include: (a) providing a solid support having a bounded feature location on a surface thereof, where the bounded feature location includes a region of the surface at least partially bounded by an electromagnetic radiation modified boundary; and (b) producing a ligand in the bounded feature location. Also provided are systems for practicing the subject methods, as well as devices produced by the methods and methods of using such devices.

As such, aspects of the invention include methods of producing a ligand feature on a surface, where a ligand is immobilized at a feature location on a surface at least partially bounded by an electromagnetic radiation-modified boundary. In certain embodiments, the methods further include producing the electromagnetic radiation-modified boundary, e.g., by laser ablation. In certain embodiments, the immobilizing includes depositing the ligand onto the feature location. In certain embodiments, the ligand is a biopolymer or precursor thereof. In certain embodiments, the ligand comprises a biopolymer precursor and multiple deposition steps are performed at the location to synthesize a biopolymer at the location. In certain embodiments, the feature location has a diameter ranging from about 10 μm to about 1 cm. In certain embodiments, the feature location is completely bounded by said electromagnetic radiation-modified boundary. In certain embodiments, the surface includes a plurality of the at least partially bounded feature locations. In certain embodiments, the plurality of the at least partially bounded feature locations form a pattern of feature locations on the surface, e.g., pattern of spots, such as an ordered pattern of columns and rows of spots. In certain embodiments, the surface has been chemically modified, e.g., by contact with at least one silanizing reagent. In certain embodiments, the surface includes silica. In certain embodiments, the immobilizing is performed at a plurality of locations to produce a ligand array. In certain embodiments, the ligand array is a nucleic acid array or a peptide array.

Also provided are systems for producing a solid support as described above, where the systems at least include an electromagnetic radiation source, e.g., a laser; and a fluid deposition element, e.g., a pulse-jet device, configured to deposit a volume of fluid that includes the ligand onto said surface. In representative embodiments, the system further includes a processor configured to operate the electromagnetic radiation source to produce a feature location on the surface.

Also provided are solid supports that include a plurality of feature locations on a surface thereof, wherein each feature location includes a region of the surface at least partially bounded by an electromagnetic radiation modified boundary.

Also provided are methods for determining whether an analyte is present in a sample, where the methods include contacting the sample with a solid support of the invention; and detecting any resultant binding complexes on said solid support to determine whether the analyte is present in said sample.

### BRIEF DESCRIPTION OF THE FIGURES

FIG. 1 illustrates a substrate carrying multiple arrays, such as may be fabricated by methods of the present invention;

FIG. 2 is an enlarged view of a portion of FIG. 1 showing multiple ideal spots or features;

FIG. 3 is an enlarged illustration of a portion of the substrate in FIG. 2; and

FIG. 4 is schematic representation depicting an apparatus in accordance with the present invention.

### DEFINITIONS

The term “polymer” means any compound that is made up of two or more monomeric units covalently bonded to each other, where the monomeric units may be the same or different, such that the polymer may be a homopolymer or a heteropolymer. Representative polymers include peptides, polysaccharides, nucleic acids and the like, where the polymers may be naturally occurring or synthetic.

“Ligands” are moieties that specifically bind to analytes of interest, where in representative embodiments ligands are polymers.

The term “peptide” as used herein refers to any polymer compound produced by amide formation between an α-carboxyl group of one amino acid and an α-amino group of another group.

The term “oligopeptide” as used herein refers to peptides with fewer than about 10 to 20 residues, i.e. amino acid monomeric units.

The term “polypeptide” as used herein refers to peptides with more than 10 to 20 residues.

The term “protein” as used herein refers to polypeptides of specific sequence of more than about 50 residues.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single-stranded nucleotide multimers of from about 10 to about 100 nucleotides and up to 200 nucleotides in length.

The term “polynucleotide” as used herein refers to single- or double-stranded polymers composed of nucleotide monomers of generally greater than about 100 nucleotides in length.

The term “functionalization” as used herein relates to modification of a solid substrate to provide a plurality of functional groups on the substrate surface. By a “functionalized surface” as used herein is meant a substrate surface that has been modified so that a plurality of functional groups are present thereon.

The terms “reactive site”, “reactive functional group” or “reactive group” refer to moieties on a monomer, polymer or substrate surface that may be used as the starting point in a synthetic organic process. This is contrasted to “inert” hydrophilic groups that could also be present on a substrate surface, e.g., hydrophilic sites associated with polyethylene glycol, a polyamide or the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise about 2-50 monomers, preferably about 2-20, more preferably about 3-10 monomers.

The term “monomer” as used herein refers to a chemical entity that can be covalently linked to one or more other such entities to form a polymer. Of particular interest to the present application are nucleotide “monomers” that have first and second sites (e.g., 5′ and 3′ sites) suitable for binding to other like monomers by means of standard chemical reactions (e.g., nucleophilic substitution), and a diverse element which distinguishes a particular monomer from a different monomer of the same type (e.g., a nucleotide base, etc.). In the art synthesis of nucleic acids of this type utilizes an initial substrate-bound monomer that is generally used as a building-block in a multi-step synthesis procedure to form a complete nucleic acid.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

As used herein, the term “amino acid” is intended to include not only the L, D- and nonchiral forms of naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine), but also modified amino acids, amino acid analogs, and other chemical compounds which can be incorporated in conventional oligopeptide synthesis, e.g., 4-nitrophenylalanine, isoglutamic acid, isoglutamine, ε-nicotinoyl-lysine, isonipecotic acid, tetrahydroisoquinoleic acid, α-aminoisobutyric acid, sarcosine, citrulline, cysteic acid, t-butylglycine, t-butylalanine, phenylglycine, cyclohexylalanine, β-alanine, 4-aminobutyric acid, and the like.

The term “alkyl” as used herein refers to substituted or unsubstituted, cyclic, or linear, branched or unbranched saturated hydrocarbon group of 1 to 24 carbon atoms. Examples include methyl, ethyl, n-propyl, isopropyl, n-butyl, isobutyl, t-butyl, octyl, decyl, tetradecyl, hexadecyl, eicosyl, tetracosyl and the like, 3-methyl-octyl, 3-methoxy-octyl, 3-chloro-octyl and the like, as well as cycloalkyl groups such as cyclopentyl, cyclohexyl and the like.

The term “alkenyl” as used herein refers to substituted or unsubstituted, cyclic, or linear, branched or unbranched unsaturated hydrocarbon group of 1 to 24 carbon atoms. Examples include octenyl, nonenyl, decenyl, undecenyl and the like, isopropenyl, isobutenyl, isopentenyl, octenyl, isoprenyl and the like. The term “alkoxy” as used herein refers to a substituent —O—R wherein R is alkyl as defined above. Examples of alkoxy groups include methoxy, ethoxy, n-propoxy, isopropoxy, sec-butoxy, tert-butoxy and the like.

The terms “halogen” or “halo” are used to refer to a chloro, bromo, fluoro or iodo substituent, or combinations thereof, such as dichloro, chlorobromo, dichlorobromo and the like.

A “silane” or “silanizing reagent” refers to a compound or reagent in which a central silicon atom is bonded to four substituents, wherein the substituents may be the same or different.

The term “protecting group” refers to chemical moieties that, while stable to the reaction conditions, mask or prevent a reactive group from participating in a chemical reaction. Protecting groups may also alter the physical properties such as the solubility of compounds, so as to enable the compounds to participate in a chemical reaction. Examples of protecting groups are known in the art, for example, Greene et al., Protective Groups in Organic Synthesis, 2nd Ed., New York: John Wiley & Sons, 1991.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

The term “sample” as used herein relates to a material or mixture of materials, typically, although not necessarily, in fluid form, containing one or more components of interest. An “array,” or “chemical array’ used interchangeably includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of ligand polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand, more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded, the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations. Each array may cover an area of less than 100 cm², or even less than 50 cm², 10 cm² or 1 cm². In representative embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays may be fabricated using drop deposition from pulse jets of either precursor units (such as nucleotide or amino acid monomers) in the case of in situ fabrication, or the previously obtained biomolecule, e.g., polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. Other drop deposition methods can be used for fabrication, as previously described herein.

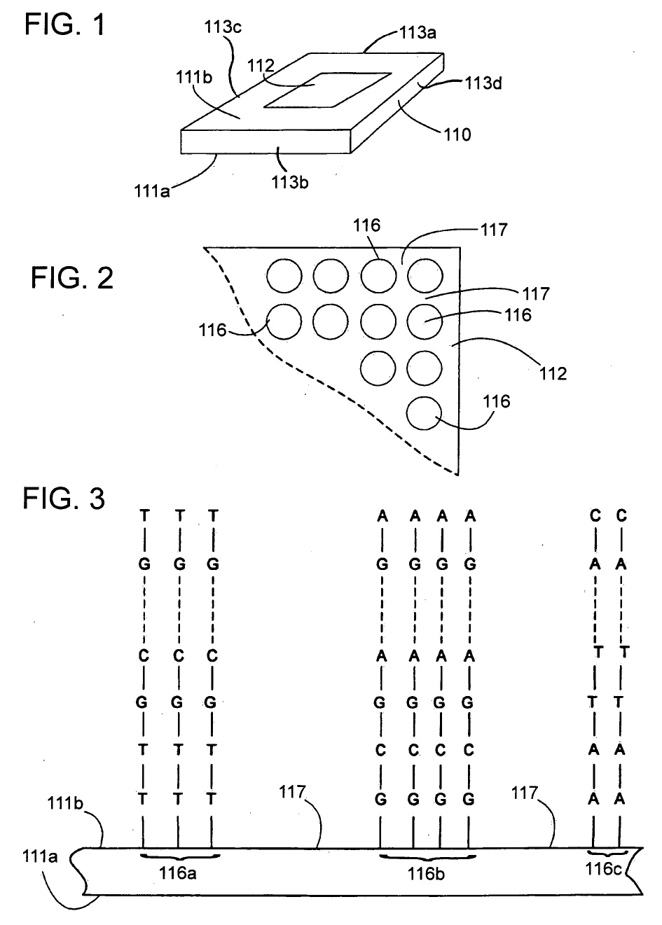

An exemplary chemical array is shown in FIGS. 1-3, where the array shown in this representative embodiment includes a contiguous planar substrate 110 carrying an array 112 disposed on a surface 111b of substrate 110. It will be appreciated though, that more than one array (any of which are the same or different) may be present on surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the surface 111b, with regions of the rear surface 111b adjacent the opposed sides 113c, 113d and leading end 113a and trailing end 113b of slide 110, not being covered by any array 112. A second surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape, as mentioned above.

As mentioned above, array 112 contains multiple spots or features 116 of biopolymer ligands, e.g., in the form of polynucleotides. As mentioned above, all of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

In those embodiments where an array includes two more features immobilized on the same surface of a solid support, the array may be referred to as addressable. An array is “addressable” when it has multiple regions of different moieties (e.g., different polynucleotide sequences) such that a region (e.g., a “feature” or “spot” of the array) at a particular predetermined location (e.g., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

An array “assembly” includes a substrate and at least one chemical array, e.g., on a surface thereof. Array assemblies may include one or more chemical arrays present on a surface of a device that includes a pedestal supporting a plurality of prongs, e.g., one or more chemical arrays present on a surface of one or more prongs of such a device. An assembly may include other features (such as a housing with a chamber from which the substrate sections can be removed). “Array unit” may be used interchangeably with “array assembly”.

The term “substrate” as used herein refers to a surface upon which ligands molecules or probes, e.g., an array, may be adhered. Glass slides are the most common substrate for biochips, although fused silica, silicon, plastic and other materials are also suitable. In representatative embodiments, the substrate includes silica.

When two items are “associated” with one another they are provided in such a way that it is apparent one is related to the other such as where one references the other. For example, an array identifier can be associated with an array by being on the array assembly (such as on the substrate or a housing) that carries the array or on or in a package or kit carrying the array assembly. “Stably attached” or “stably associated with” means an item's position remains substantially constant where in certain embodiments it may mean that an item's position remains substantially constant and known.

A “web” references a long continuous piece of substrate material having a length greater than a width. For example, the web length to width ratio may be at least 5/1, 10/1, 50/1, 100/1, 200/1, or 500/1, or even at least 1000/1.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

“Rigid” refers to a material or structure which is not flexible, and is constructed such that a segment about 2.5 by 7.5 cm retains its shape and cannot be bent along any direction more than 60 degrees (and often not more than 40, 20, 10, or 5 degrees) without breaking.

“Hybridizing” and “binding”, with respect to polynucleotides, are used interchangeably. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

“Stringent hybridization conditions” and “stringent hybridization wash conditions” in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions sets forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

“Contacting” means to bring or put together. As such, a first item is contacted with a second item when the two items are brought or put together, e.g., by touching them to each other.

“Depositing” means to position, place an item at a location-or otherwise cause an item to be so positioned or placed at a location. Depositing includes contacting one item with another. Depositing may be manual or automatic, e.g., “depositing” an item at a location may be accomplished by automated robotic devices.

By “remote location,” it is meant a location other than the location at which the array (or referenced item) is present and hybridization occurs (in the case of hybridization reactions). For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information means transmitting the data representing that information as signals (e.g., electrical, optical, radio signals, and the like) over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

An array “package” may be the array plus only a substrate on which the array is deposited, although the package may include other features (such as a housing with a chamber).

A “chamber” references an enclosed volume (although a chamber may be accessible through one or more ports). It will also be appreciated that throughout the present application, that words such as “top,” “upper,” and “lower” are used in a relative sense only.

It will also be appreciated that throughout the present application, that words such as “cover”, “base” “front”, “back”, “top”, are used in a relative sense only. The word “above” used to describe the substrate and/or flow cell is meant with respect to the horizontal plane of the environment, e.g., the room, in which the substrate and/or flow cell is present, e.g., the ground or floor of such a room.

### DETAILED DESCRIPTION OF REPRESENTATIVE EMBODIMENTS

Methods and compositions for producing a solid support having a ligand immobilized on a surface thereof, e.g. a ligand array, are provided. Aspects of the methods include: (a) providing a solid support having a bounded feature location on a surface thereof, where the bounded feature location includes a region of the surface at least partially bounded by an electromagnetic radiation modified boundary; and producing a ligand in the bounded feature location. Also provided are systems for practicing the subject methods, as well as devices produced by the methods and methods of using such devices.

Before the present invention is described in greater detail, it is to be understood that this invention is not limited to particular embodiments described, as such may, of course, vary. It is also to be understood that the terminology used herein is for the: purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to Which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized above, in one aspect, the invention provides methods for producing a solid support (e.g., a substrate) having a ligand immobilized on the surface thereof. A feature of embodiments of the subject invention is that the ligand is present as a ligand feature, where the ligand feature is made up of a region of immobilized ligand at least partially bounded by an electromagnetic radiation surface modified boundary. The subject methods include providing a substrate having a surface that has been modified with an electromagnetic radiation surface modification protocol, for instance, by laser ablation, or the like, so as to produce one or more bounded feature locations on the surface of the substrate. In accordance with the methods of the subject invention, the surface of the substrate may be functionalized prior to the bounded feature location generation step, e.g. in a surface energy modification protocol. Following the bounded feature location generation slip, a surface immobilized ligand may then be produced in the bounded feature location to produce a desired ligand feature. Other aspects of the invention include arrays produced by the substrate methods, systems for producing the arrays, methods of using the subject arrays and kits that include the subject arrays. Each of these different aspects of the different embodiments of the invention is now described in greater detail below.

In the subject methods, an aspect is to provide a solid support (e.g., a substrate) that has a surface which includes a bounded feature location. By bounded feature location is meant a region of a substrate that is a least partially bounded (i.e., bordered) by a modified surface boundary, where the surface has been modified using an electromagnetic radiation surface modification protocol.

An electromagnetic radiation surface modification protocol is a protocol for modifying a surface of a solid support that uses electromagnetic radiation to modify the surface. Any suitable source for delivering electromagnetic radiation may be used so long as it is capable of delivering a desired quantity of energy and/or momentum (e.g., one or more photons) to a specific location on the surface of the substrate and thereby produce a modified surface, e.g. an ablated or etched surface. In representative embodiments, the modified boundary produced by this protocol has a surface energy that is different from the surface energy of the feature locations bounded by the modified boundary.

In a representative embodiment, laser ablation is used to deliver a precise quantity of energy to the surface of a substrate to remove material from the substrate and/or a coating thereon. Laser ablation may be performed by any means well known in the art, but typically involves the use of a high-energy photon laser such as an excimer laser. There are several types of excimer lasers that may be used, for instance: F2, ArF, KrCl, KrF, XeBr, XeCl or XeF lasers. Suitable excimer lasers are available for purchase from XMR Inc. (Santa Clara, Calif.), or Lambda Physik GMbH (Gottingen, Germany), etc. However, any excimer laser may be used so long as it is capable of delivering a narrow beam of ultraviolet light that is intense enough to photodissociate the chemical bonds in the substrate surface, a chemical coating on the surface of the substrate (silanation, photosensitive emulsion, photoresist, etc.) or both, in a dimensionally precise manner and thereby create a defined location on the surface of the substrate. Laser ablation techniques are well known in the art and described, for example, by Znotins et. Al. (1987) Laser Focus Electro Optics, at pp. 54-70, and in U.S. Pat. Nos. 5,291,226 and 5,305,015 to Schantz et al.

Other ultraviolet light sources with substantially the same optical wavelengths and energy densities (e.g., fluence) may be used as well, so long as they are capable of delivering short pulses of intense ultraviolet light energy that can be absorbed by a thin surface layer of the substrate, a substrate coating, or both, and are therefore capable of modifying a discrete location and/or pattern on the surface of the substrate. In order to ensure precise removal and shaping of the material to be ablated, the energy density of the laser source is above the fluence threshold of the material in certain embodiments. For instance, the absorbed ultraviolet light energy may be concentrated in a small enough volume of material that it is rapidly heated, dissociated, and ejected away from the surface of the substrate. Additionally, the pulse length may be sufficiently small (and repetition rate sufficiently high) enough so as to produce the desired ablation without the propagation of heat to the surrounding material, so that the surrounding region is not melted or otherwise damaged.

Accordingly, the type of laser used and the energy density, pulse duration, and repetition rate of the emitted ultraviolet light pulses will vary depending on the material to be ablated and its fluence threshold. One of ordinary skill in the art can readily determine these parameters based on the objective to be achieved. In one representative embodiment, the wavelength of the light emitted from the laser source is in the ultraviolet region, for instance ultraviolet light with a wavelength from about 150 nm to about 375 nm, including 175 nm to about 325 nm, and from 225 nm to about 200 nm. Suitable pulse energies, in representative embodiments range from about 100 millijoules per square centimeter to about 1 joule per square centimeter. Suitable pulse rates in representative embodiments range from about 10 Hz to about 10 kHz, from about 100 Hz to about 1 kHz, and about 250 Hz to about 500 Hz. Suitable pulse durations in representative embodiments range from about 1 ns to about 1 ms, and about 10 ns to about 500 ns, including about 20 ns to about 100 ns, and about 30 ns to 60 ns. The total number of pulses delivered may vary and in representative embodiments ranges between 1 pulse and about 1000 pulses, about 10 pulses and about 500 pulses, including about 50 pulses and about 250 pulses. Any of these parameters can be easily adjusted and controlled, by means well known in the art, so as to ablate precise locations upon the surface of the solid support.

As summarized above, a bounded feature location is a region of a surface that is at least partially bounded (i.e., bordered) by a boundary region. The boundary region is a region of the surface that has been modified by electromagnetic radiation, e.g., laser ablation, as described above. By at least partially bounded is meant that at least about 10% or more, such as at least about 25% or more, including at least about 50% or more, such as at least about 75% or more, e.g. at least about 90% or more of the perimeter of the feature region is bounded by the modified surface boundary. In representation embodiments, the entire feature region is surrounded with the boundary. For example, in those embodiments where the feature location is a circular spot, the interior circular region of the feature is encircled by the boundary.

The width of a given boundary region may vary so long as it is sufficiently wide to retain fluid deposited onto the feature region within the feature region. In representative embodiments the width of a given boundary ranges from about 1 μm to about 50 μm, such as from about 5 μm to about 25 μm.

Embodiments of the invention are characterized in that the solid support surface includes a plurality of bounded feature locations, where by plurality is meant 2 or more, such as about 10 or more, including about 50 or more, etc., where in representative embodiments the surface includes 100 or more, 1000 or more, 5000 or more, 10,000 or more, 25,000 or more bound feature locations. The density of bounded feature locations in such embodiments may vary, and maybe at least about 10/cm², such as at least about 50/cm², including at least about 100/cm² e.g., at last about 400/cm², 1000/cm² or denser. The array of bounded feature locations may be in a precise pattern, such as a plurality of spots in an ordered pattern of columns and rows.

In certain embodiments, the solid support whose surface is modified to produce one or more bounded feature locations is a solid support that has a surface which has been chemically modified (e.g., functionalized) with a surface energy modification reagent. Accordingly, in a representative embodiment, the surface of a solid support (e.g., a substrate) is first functionalized by being contacted with a surface energy modification reagent, such as a silane derivatizing composition that contains one or more types of silanes, and is then modified by an electromagnetic radiation surface modification protocol, so as to produce a surface with at least one bounded feature location. A feature of this embodiment is that the bounded region of the bounded feature location retains the surface properties imparted to the surface in the surface energy modification step. In these embodiments, electromagnetic radiation (e.g., laser ablation) may be used to ablate the silane coating so as to form the outer boundary of a bounded feature location.

In these particular embodiments, the surface of the solid support is derivatized by contacting it with a silane derivatizing composition of one or more silanizing reagents. In certain embodiments of interest, the derivatizing composition may include two or more types of silanes, which may be the same or different from one another. For instance, the two or more silanes may differ with respect to their leaving group substituents, which may include, but are not limited to: halogens, chloro, alkoxy, aryloxy moieties, lower alkyl, e.g., methyl, ethyl, isopropyl, n-propyl, t-butyl moieties, and the like. In those embodiments where a mixture of silanes make up the derivatizing composition, the first silane is a derivatizing agent that reduces surface energy as desired, while the second silane provides a desired functionality.

For instance, the second silane may include a functional group that can bind directly to an additional molecular species of interest, or a modifiable group that can be converted to a functional group under conditions that do not substantially affect any other chemical species that are present. The functional group may be any group that facilitates the binding of a ligand to the substrate such as, but not limited hereto a hydroxyl, carboxyl, amino, or the like, or it may be a modifiable group such an olefinic moiety, e.g., a terminal CH═CH₂ group, which can readily be converted to a reactive hydroxyl group by hydroboration and oxidation using procedures known in the art. Additional functional groups of interest include, but are not limited to, those described in U.S. Pat. Nos. 6,660,338; 6,444,268; 6,387,631; 6,319,674; 6,291,183; and 6,258,454. Methods for silanizing a solid support are well known in the art, for instance, see co-pending U.S. application Ser. No. 11/050,139, which is incorporated in its entirety herein by reference. See also U.S. Pat. No. 6,258,454 for a further description of the general process of derivatizing a surface, the disclosure of which is herein incorporated by reference.

Where desired, the functionalized surface may be further functionalized prior to electromagnetic radiation modification and ligand attachment. For example a functional group (e.g., hydroxyl group) may be converted to a number of different types of functional groups which are reactive to the ligand (or precursor thereof) of interest, i.e., ligand reactive functional groups. By ligand reactive functional groups is meant groups that react with moieties present on the target ligands, (i.e., the ligands to be deposited onto the surface and covalently bound thereto) in a manner that produces a covalent bond or linkage between the ligand and the substrate surface.

A functional group(s) may be converted to a variety of different types of reactive moieties using a variety of different protocols, depending on the particular nature of the ligand that is to be covalently bound to the substrate surface. Where the functional group is a hydroxyl functional group, representative ligand reactive functional groups to which the initial hydroxyl functional groups may be converted include: aldehydes, and the like. The particular ligand reactive functional group to which the initial functional group is converted will be chosen, at least in part, on considerations that include, but are not limited to: the nature of the ligand and functional groups that may be present thereon, ease of conversion, and the like. The particular conversion protocol employed will vary with respect to the nature of the desired ligand reactive functional group, and may or may not involve the production of one or more intermediate groups. In one embodiment, the hydroxyl functional groups of the initial substrate surface are converted to aldehyde functional groups, e.g., via controlled oxidation to alde hyde functionalities, e.g., via Moffat oxidations, where primary alcohols are specifically and efficiently converted to the corresponding aldehydes under mild conditions. See e.g., Pftizner and Moffatt, Comp. Org Syn. 7, 291 (1991), J. Amer. Chem. Soc. (1965) 87:5670-78. In yet another embodiment, the surface hydroxyl groups are converted to amine reactive benzaldehyde functionalities using benzaldehyde phosphoramidites. More specifically, the hydroxyl moiety can be reacted with a benzaldehyde phosphoramidite, followed by acidic deprotection of the benzaldehyde moiety and basic deprotection of the phosphate moiety. Such protocols are known in the art, see e.g., WO 01/09385 and its priority application Ser. No. 09/364,320, the disclosure of latter of which is herein incorporated by reference. Once the surface of the substrate has been further functionalized, as desired, it may then be modified with an electromagnetic radiation protocol, as set forth above, to create boundary demarcated features on the surface of the solid support to which ligands may be attached.

Following production of the desired solid support having one or more bounded feature locations, as described above, the resultant substrate can be employed in the fabrication of solid supports having ligands immobilized on a surface thereof, e.g. such as ligand arrays. In making such structures, ligands are produced in the bounded regions of the bounded feature locations, e.g., via polymeric ligand deposition (where one or more pre-made polymeric ligands are contacted with the modified surface); or in-situ polymeric ligand synthesis, as described immediately below in greater detail.

In representative embodiments of interest, the ligands produced in the bounded feature locations are polymeric binding agents. The polymeric binding agents may vary widely, where the only limitation is that the polymeric binding agents are made up of two or more, usually a plurality of, monomeric units covalently attached in sequential order to one another such that the polymeric compound has a sequence of monomeric units. Typically, the polymeric binding agent includes at least 5 monomeric units, usually at least 10 monomeric units and more usually at least 15 monomeric units, where in many embodiments the number of monomeric units in the polymers may be as high as 5000 or higher, but generally will not exceed about 2000. In certain embodiments, the number of monomeric residues in the polymeric binding agent is at least about 50, usually at least about 100 and more usually at least about 150.

Pre-made polymeric binding agents of particular interest include biopolymeric molecules, such as polypeptides, nucleic acids, polysaccharides and the like, where polypeptides and nucleic acids, as well as synthetic mimetics thereof, are of particular interest in many embodiments.

In representative embodiments, the polymeric binding agents are nucleic acids, including DNA, RNA, nucleic acids of one or more synthetic or non-naturally occurring nucleotides, and the like. The nucleic acids may be oligonucleotides, polynucleotides, including cDNAs, mRNAs, peptide-nucleic acids and the like. Where the polymeric compounds are nucleic acids, the nucleic acids will generally be at least about 5 nt, usually at least about 10 nt and more usually at least about 15 nt in length, where the nucleic acids may be as long as 5000 nt or longer, but generally will not exceed about 3000 nt in length and usually will not exceed about 2000 nt in length. In representative embodiments, the nucleic acids are at least about 25 nt in length, usually at least about 50 nt in length and may be at least about 100 nt in length.

In certain embodiments where premade ligands are deposited onto bounded feature locations, the ligands are characterized by having a functional moiety that reacts with the ligand reactive functional moiety present in the bounded feature location to produce a covalent bond between the ligand and the substrate surface. The ligand may naturally include the desired reactive functionality, or may be modified to include the desired reaction functionality. Representative reactive functionalities of interest include, but are not limited to: amine groups, hydroxyl groups, sulfhydryl, phosphoramidite, anhydrides, and the like.

In representative embodiments, at least two distinct polymers are contacted with at least two distinct bounded feature locations, e.g., produced as described above. By distinct is meant that the two polymers differ from each other in terms of sequence of monomeric units. The number of different polymers that are contacted with the substrate surface may vary depending on the desired nature of the array to be produced, i.e. the desired density of polymeric structures. Generally, the number of distinct polymers that are contacted with the surface of the array will be at least about 5, usually at least about 10 and more usually at least about 100, where the number may be as high as 1,000,000 or higher, but in many embodiments will not exceed about 500,000 and in certain embodiments will not exceed about 100,000.

The polymers are generally contacted with the surface in an aqueous solvent, such that aqueous conditions are established at the surface location to which the polymer is to be covalently attached. The temperature during contact typically ranges from about 10 to about 60 and usually from about 20 to about 40° C. Following initial contact, the aqueous solution of polymer is typically maintained for a period of time sufficient for the desired amount of reaction to occur, where the period of time is typically at least about 20 sec, usually at least about 1 min and more usually at least about 10 min, where the period of time may be as great as 20 min or greater.

Each polymer is typically contacted with the substrate surface as part of an aqueous composition, i.e., an aqueous composition of the polymer in an aqueous solvent is contacted with the surface of the array. The aqueous solvent may be either water alone or water in combination with a co-solvent, e.g. an organic solvent, and the like. The aqueous composition may also contain one or more additional agents, including: acetic acid, monochloro acetic acid, dichloro acetic acid, trichloro acetic acid, acetonitrile, catalysts, e.g. lanthanide (III) trifluoromethylsulfate, lithium chloride, buffering agents, e.g. sodium phosphate, salts, metal cations, surfactants, enzymes, etc.

The aqueous polymer composition may be contacted with the surface using any convenient protocol. Generally, the aqueous polymer composition is contacted with the surface by depositing the aqueous polymer composition on the surface of the substrate. The aqueous volume may be deposited manually, e.g. via pipette, or through the use of an automated machine or device. A number of devices and protocols have been developed for depositing aqueous solutions onto precise locations of a support surface and may be employed in the present methods. Such devices include “pulse-jet” printing devices, mechanical deposition or pipetting devices and the like. See e.g. U.S. Pat. Nos. 4,877,745; 5,338,688; 5,474,796; 5,449,754; 5,658,802; 5,700,637; and 5,807,552; the disclosures of which are herein incorporated by reference. Robotic devices for precisely depositing aqueous volumes onto discrete locations of a support surface, i.e. arrayers, are also commercially available from a number of vendors, including: Genetic Microsystems; Cartesian Technologies; Beecher Instruments; Genomic Solutions; and BioRobotics, to name representative vendors.

The amount of fluid that is deposited may vary. For example, volumes ranging from about 1 nl to 1 pl, such as from about 60 to 100 pl may be deposited onto the substrate surface. Following contact and incubation for a period of time and under conditions sufficient for the desired reaction to occur, as described above, the surface of the resultant array may be further processed as desired in order to prepare the array for use, as described below. As such, the array surface may be washed to remove unbound reagent, e.g., unreacted polymer, and the like. Any convenient wash solution and protocol may be employed, e.g., flowing an aqueous wash solution, e.g., water, methanol, acetonitrile, and the like, across the surface of the array, etc. The surface may also be dried and stored for subsequent use, etc.

Because of the nature of the bounded feature locations, fluid deposited onto the bounded feature locations stays within the boundary, such that precise features of controlled dimensions are produced. The above-described protocols for array fabrication can be carried out using the devices described in U.S. Pat. Nos. 6,242,266; 6,232,072 and 6,180,351.

As indicated above, the modified and/or functionalized substrate surfaces produced as described above can also be employed in in-situ ligand synthesis applications. The in-situ synthesis methods include those described in U.S. Pat. No. 5,449,754 for synthesizing peptide arrays, as well as WO 98/41531 and the references cited therein for synthesizing polynucleotides (specifically, DNA) using phosphoramidite or other chemistry. Further details of in situ methods are provided in U.S. Pat. No. 6,242,266, U.S. Pat. No. 6,232,072, U.S. Pat. No. 6,180,351, U.S. Pat. No. 6,171,797, U.S. Pat. No. 6,323,043, and U.S. patent application Ser. No. 09/302,898.

The above protocol produces chemical, e.g., ligand, arrays that can be employed in a variety of different applications, as described in greater detail below.

Whether the ligands are deposited onto the surface of the array in premade form or produced on the surface in:situ by deposition of precursors thereof, a common step to both approaches is the production of the desired two or more ligands on the functionalized surface. A feature of certain embodiments is that two or more different ligands or precursors thereof are deposited (e.g., by pulse-jet deposition) onto discrete bounded feature locations.

The invention also provides arrays of polymeric binding agents produced according to the methods described above. The arrays include at least two distinct polymers that differ by monomeric sequence immobilized on, e.g., covalently bonded to, different and known locations on the substrate surface. In certain embodiments, each distinct polymeric sequence of the array is typically present as a composition of multiple copies of the polymer on the substrate surface, e.g., as a spot on the surface of the substrate. The number of distinct polymeric sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/feature from another.

In the broadest sense, the arrays of the invention are arrays of ligand, and generally, polymeric binding agents, where the polymeric binding agents may be any of: peptides, proteins, nucleic acids, polysaccharides, synthetic mimetics of such biopolymeric binding agents, etc. In representative embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini, e.g., the 3′ or 5′ terminus. In other embodiments, the arrays are arrays of polypeptides, e.g., proteins or fragments thereof.

In certain embodiments, the arrays produced according to the subject methods are in situ produced high resolution arrays, where by high resolution is meant that the density of the individual features have a high density. By high density is meant at least about 100 features/cm², usually at least about 500 features/cm², where the density may, in certain embodiments, range from about 500 to about 10,000 or more, such as from about 500 to about 10,000 features/cm². This high resolution feature is achievable using in situ preparation protocols particular in those embodiments where the substrate surface is a pattern functionalized surface, as described above.

A feature of the arrays produced according to the subject methods is that the size and dimensions of the features are highly controlled, and in representative embodiments, uniform. As such, in representative embodiments where the features are uniform, any variation in spot size diameter of any two features on the array (or at least any two bounded features) does not differ by more than about 10 μm, such as by no more than about 5 μm, including by more than about 2.5 μm. In addition, the features of the array include a region to which the ligand is immobilized, where the ligand displaying region is bounded (at least partially) by the electromagnetic radiation modified boundary or border. In yet other embodiments where the features are not uniform, because of the manner in which the arrays are made, the configuration or shape of the features is highly controlled, such that there is little if any variation between the shape of the actual feature and the intended shape of the feature. As such, arrays produced according to the present methods are readily distinguished from arrays produced according to other methods known in the art.

The subject invention also provides apparatuses (i.e., systems) for practicing the subject methods, as reviewed above. In representative embodiments, the systems include: (a) a source of electromagnetic radiation, e.g., a laser; and (b) a fluid deposition element for depositing a volume of fluid onto a bounded feature location on the surface of solid support. In certain embodiments the source of electromagnetic radiation is an excimer laser. Additionally, in certain embodiments the fluid deposition element is a pulse jet.

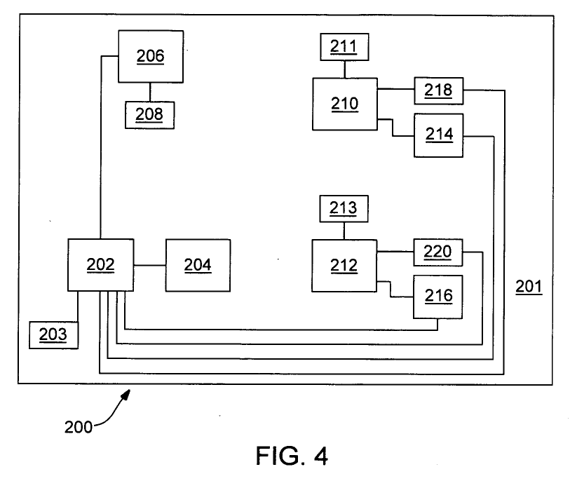

One representative embodiment of an apparatus in accordance with the present invention is depicted in FIG. 4 in schematic form. Apparatus 200 includes platform 201 on which the components of the apparatus are mounted. Apparatus 200 includes main computer 202, with which various components of the apparatus are in communication. Video display 203 is in communication with computer 202. Apparatus 200 further includes fluid deposition element 204, which is controlled by main computer 202. The nature of fluid deposition element 204 depends on the nature of the deposition technique employed to add fluid to the substrate surface. Such deposition techniques include, by way of illustration and not limitation, printing techniques, such as pulse-jet deposition printing, and so forth. Transfer robot 206 is also controlled by main computer 202 and includes a robot arm 208 that moves a substrate from electromagnetic radiation modification platform 210 to fluid deposition element 204, or to any other position such as to and/or from a functionalization or other reaction chambers (not shown).

In one embodiment robot arm 208 introduces a substrate horizontally to electromagnetic radiation modification platform 210 for modification of the substrate surface by an electromagnetic radiation protocol so as to generate one or more bounded feature locations on the surface of the substrate, e.g. according to a predetermined set of instructions provided by computer 202, e.g. as may be present in a memory of the computer in a pattern file and implemented bu a processor. Arm 208 then introduces the substrate into fluid deposition element 204 for depositing a fluid droplet in the one or more bounded feature locations on the substrate surface. Mechanisms for rotating a substrate are well known in the art and include, but are not limited to, pneumatic pistons, belt or chain drives, cams and followers, rack and pinions or other gear drives, lead screws, direct drive motors, etc, which may be controlled by a processor.

Electromagnetic radiation modification platform 210 is in communication with program logic controller 214 which corresponds to a controller (not shown), which is controlled by main computer 202. Electromagnetic radiation modification platform is in communication with electromagnetic radiation source 211 and sensor indicator 218, which are also controlled by main computer 202. Main computer 202 controls program logic controller 214, which in turn controls both the electromagnetic radiation modification platform 210 and the electromagnetic radiation source 211, and is capable of controlling the movement of one with respect to the other so as to generate a desired pattern of spot boundary feature locations in accordance with a predetermined size, shape and pattern.

In some embodiments, the apparatus of the invention may optionally include one or more additional reaction chambers for contacting the substrate surface with a washing agent, an oxidizing agent, a capping agent, a deblocking agent, or the like. For example, the subject apparatus may include first, second, third, etc. different reaction chambers for contacting the substrate surface with one or more of the washing, oxidizing, capping, deblocking agents, or the like. The apparatus of the invention further includes appropriate electrical and mechanical architecture and electrical connections, wiring and devices such as timers, clocks, and so forth for operating the various elements of the apparatus.

The methods in accordance with the present invention may be carried out under computer control, that is, with the aid of a computer. The computer may be driven by software specific to the methods described herein. Examples of software or computer programs used in assisting in conducting the present methods may be written in any convent language, e.g. Visual BASIC, FORTRAN and C++ (PASCAL, PERL or assembly language). It should be understood that the above computer information and the software used herein are by way of example and not limitation.

In one aspect, the present invention is directed to a computer program that may be utilized to carry out the above method steps. The computer program provides for controlling the program logic controller 214 and the fluid deposition element 204. The computer program includes a readable storage medium that has instructions for reading and generating a desired pattern of feature locations and instructions for directing the logic controller 214 so as to generate a surface modified substrate in accordance with the present invention. The computer program further has instructions for controlling the fluid deposition element 204 and thereby directs the deposition of a ligand, or ligand precursor, to the precise pattern of feature locations generated by the electromagnetic radiation protocol. In this way a ligand array may be produced in accordance with the methods of the subject invention. The computer program also includes elements for communicating the appropriate instructions to the appropriate elements.

Another aspect of the present invention is a computer program product including a computer readable storage medium having a computer program stored thereon which, when loaded into a computer, performs the aforementioned method. In exemplary embodiments, the methods are coded onto a computer-readable medium in the form of programming. The data storage means may include any manufacture including a recording of the present information as described above, or a memory access means that can access such a manufacture.

In certain embodiments, a processor of the subject invention may be in operable linkage, i.e., part of or networked to, the aforementioned device, and capable of directing its activities. A processor may be pre-programmed, e.g., provided to a user already programmed for performing certain functions, or may be programmed by a user, where a processor may be programmed, e.g., by a user, from a remote location meaning a location other than the location at which the processor and/or flow cell and/or substrate is present. For example, a remote location could be another location (e.g. office, lab, etc.) in the same city, another location in a different city, another location in a different state, another location in a different country, etc. A processor may be remotely programmed by “communicating” programming information to the processor, i.e., transmitting the data representing that information as electrical signals over a suitable communication channel (for example, a private or public network). Any convenient telecommunications means may be employed for transmitting the programming, e.g., facsimile, modem, Internet, LAN, WAN or other network means, wireless communication, etc.

Ligand arrays produced as described above find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g. an isotopic or fluorescent label present on the analyte, etc. The presence of the analyte in the sample is then deduced from the detection of binding complexes on the substrate surface.

Specific analyte detection applications of interest include assays in which the biopolymeric solid supports (e.g., nucleic acid arrays) of the subject invention are employed. In these assays, a sample of analyte target molecules (e.g., nucleic acids) is first prepared, where preparation may include labeling of the target with a label, e.g. a member of signal producing system. Following sample preparation, the sample is contacted with a modified ligand solid support produced by the methods of the subject invention, wherein the solid support contains at least one defined electromagnetic radiation produced feature that includes an immobilized ligand therein. The sample is contacted with the solid support under conditions (e.g., hybridization conditions) that allow an analyte in the, sample to bind to a surface immobilized ligand that is specific for that analyte, and thereby to form a complex between the analyte (e.g., nucleic acid) and the attached ligand (e.g., nucleic acid complementary to the analyte sequence). The presence of any resultant binding complexes of the ligand and the analyte is then detected to determine whether the analyte is present in the sample.

In one representative embodiment an analyte detection assay is a hybridization assay. Specific hybridization assays of interest which may be practiced using the subject arrays include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992. Also of interest are U.S. Pat. Nos. 6,656,740; 6,613,893; 6,599,693; 6,589,739; 6,587,579; 6,420,180; 6,387,636; 6,309,875; 6,232,072; 6,221,653; and 6,180,351.

Hybridization assays can have a variety of applications, e.g., detection of expressed sequences, diagnosis, genotyping, detection of nucleic acid copy number (e.g., in an array CGH assay), detection of binding sites in genomic DNA or in RNA of proteins or other molecules (e.g., such as in a location analysis assay). As used herein, “detection of an analyte” encompasses all of these applications. Further, in certain embodiments, “detection of an analyte” may involve evaluating the hybridization of multiple different analytes, e.g., to determine the amount of one molecule in a sample capable of binding to a ligand at a feature relative to other molecules in the sample. In certain aspects, the different molecules can be differentially labeled and “detection” of an analyte may include determining a relative amounts or ratios of differentially labeled sample molecules.

Where the arrays are arrays of polypeptide binding agents, e.g., protein arrays, specific applications of interest include analyte detection/proteomics applications, including those described in U.S. Pat. Nos. 4,591,570; 5,171,695; 5,436,170; 5,486,452; 5,532,128 and 6,197,599 as well as published PCT application Nos. WO 99/39210; WO 00/04832; WO 00/04389; WO 00/04390; WO 00/54046; WO 00/63701; WO 01/14425 and WO 01/40803—the disclosures of which are herein incorporated by reference.

As such, in using an array made by the methods of the present invention, the array will typically be exposed to a sample (for example, a fluorescently labeled analyte, e.g., nucleic acid-containing or protein-containing sample) and the array can then read (e.g., binding complexes can be detected). Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as signals (e.g., electrical, optical, radio signals, and the like) over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

Also provided are kits for use in practicing methods of the invention. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like.

The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay.

The kits may also include instructions that may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of producing a ligand feature on a surface, said method comprising:
immobilizing a ligand at a feature location on a surface at least partially bounded by an electromagnetic radiation-modified boundary.

2. The method according to claim 1, further comprising producing said electromagnetic radiation-modified boundary.

3. The method according to claim 1, wherein said electromagnetic radiation modified boundary is a laser ablation-produced boundary.

4. The method according to claim 1, wherein said immobilizing comprises depositing the ligand onto said feature location.

5. The method according to claim 1, wherein said ligand is a biopolymer or precursor thereof.

6. The method according to claim 4, wherein the ligand comprises a biopolymer precursor and multiple deposition steps are performed at the location to synthesize a biopolymer at the location.

7. The method according to claim 1, wherein said feature location has a diameter ranging from about 10 μm to about 1 cm.

8. The method according to claim 1, wherein said feature location is completely bounded by said electromagnetic radiation-modified boundary.

9. The method according to claim 1, wherein said surface comprises a plurality of said at least partially bounded feature locations.

10. The method according to claim 9, wherein said plurality of said at least partially bounded feature locations form a pattern of feature locations on said surface.

11. The method according to claim 10, wherein said pattern of feature locations is a pattern of spots.

12. The method according to claim 11, wherein said pattern of spots comprises an ordered pattern of columns and rows of spots.

13. The method according to claim 1, further comprising producing said electromagnetic radiation-modified boundary by laser ablation.

14. The method according to claim 12, wherein said surface has been chemically modified.

15. The method according to claim 14, wherein said chemically-modified surface has been contacted with at least one silanizing reagent.

16. The method according to claim 1, wherein said surface comprises silica.

17. The method according to claim 1, wherein said immobilizing is performed at a plurality of locations to produce a ligand array.

18. The method according to claim 17, wherein said ligand array is a nucleic acid array.

19. The method according to claim 17, wherein said ligand array is a peptide array.

20. A system for producing a solid support having a ligand immobilized on a surface thereof, said system comprising:
(a) an electromagnetic radiation source; and
(b) a fluid deposition element configured to deposit a volume of fluid comprising said ligand onto said surface.

21. The system according to claim 20, wherein said electromagnetic radiation source comprises a laser.

22. The system according to claim 20, wherein said fluid deposition element comprises a pulse jet device.

23. The system according to claim 20, wherein said system further comprises a processor configured to operate said electromagnetic radiation source to produce a feature location on said surface.

24. A solid support comprising a plurality of feature locations on a surface thereof, wherein each feature location comprises a region of said surface at least partially bounded by an electromagnetic radiation modified boundary.

25. The solid support according to claim 24, wherein said electromagnetic radiation modified boundary is produced by laser ablation.

26. The solid support according to claim 24, wherein said feature location comprises an immobilized ligand.

27. The solid support according to claim 26, wherein said solid support comprises a plurality of feature locations having immobilized ligands.

28. The solid support according to claim 26, wherein said ligand is a biopolymer or precursor thereof.

29. The solid support according to claim 28, wherein said biopolymer comprises a nucleic acid molecule.

30. The solid support according to claim 28, wherein said biopolymer comprises a peptide.

31. A method for determining whether an analyte is present in a sample, said method comprising:
a) contacting said sample with a solid support according to claim 26; and
b) detecting any resultant binding complexes on said solid support to determine whether said analyte is present in said sample.
